(19) United States
(12) Patent Application Publication
(10) Pub. No.: US 2006/0083795 A1
(43) Pub. Date: Apr. 20, 2006

(54) Meal replacement products having appetite suppressing qualities

(76) Inventors: Lima Shatkina, Orem, UT (US); Rufina Shatkina, Orem, UT (US); Sam Gurevich, Orem, UT (US)

Correspondence Address: Bryan G. Pratt;Rader, Fishman & Grauer, 10653 S. River Front Parkway, South Jordan, UT, 84095, US

(21) Appl. No.: 11/253,234

(22) Filed: Oct. 17, 2005

Related U.S. Application Data
(60) Provisional application No. 60/620,195, filed on Oct. 19, 2004.

(51) Int. Cl. A61K 36/185

(52) U.S. Cl. 424/725; 424/769

(57) ABSTRACT

A meal replacement is provided herein. According to one exemplary embodiment, the meal replacement includes a meal replacement base, and an appetite suppressant including a succulent plant extract. According to one exemplary embodiment, the succulent plant extract is selected from the group consisting of Trichocaulon piliferum, Trichocaulon officinale, Hoodia gordonii, Hoodia currorii, Hoodia lugardii, and the Caralluma group of plants.

### RELATED APPLICATION

The present application is related to a previously filed U.S. provisional patent application entitled “Meal Replacement Product Having Appetite Suppressing Qualities,” filed Oct. 19, 2004 and having a Ser. No. 60/620,195, the contents of which are hereby incorporated by reference in their entirety.

### BACKGROUND

Drugs are sometimes used to lower food intake by suppressing appetite. Centrally acting appetite suppressant drugs used in the treatment of obesity fall into two broad pharmacological categories; those which act via brain catecholamine pathways and those which act via serotonin pathways. Of the group that act via brain catecholamine, amphetamine and phenmetrazine are no longer recommended because of their stimulant properties and addictive potential. Some of the remaining drugs in the group that act via brain catecholamine include amfepramone (diethylpropion), phentermine, mazindol and phenylpropanolamine. Most, if not all, of these remaining drugs have been shown to reduce appetite and lower food intake, thereby helping obese patients more easily keep to a low-calorie diet and lose weight. Further, most if not all of these remaining drugs have some sympathomimetic and stimulant properties that produce effects similar to the “fight or flight” response, which cause increased heart rate, sweating, and increased blood pressure.

Other drugs which act via serotonin pathways may not have such effects. In particular, anorectic drugs which promote serotonin neurotransmission may not have such stimulant or sympathomimetic properties. Drugs which act via serotonin pathways may include fenfluramine, together with its recently introduced dextrorotatory stereoisomer dexfenfluramine, and fluoxetine. These drugs reduce appetite and food intake and are effective in the treatment of obesity. Anorectic drugs are frequently reserved for those who are clinically at risk from being overweight, and then as part of a comprehensive weight-reducing program including regular dietary counseling. Although current licensing regulations only allow their use over a relatively short period, such as about twelve to sixteen weeks, clinical trials have shown that these drugs may be effective over longer periods, particularly in preventing weight regain. Of the compounds currently indicated for use in obesity, dexfenfluramine appears to have the most suitable pharmacological profile, although it should not be given to patients with a history of depression. Meal replacements are meant to be taken instead of a meal, but unlike after a meal, one is typically still hungry after a meal replacement.

### SUMMARY

A meal replacement is provided herein. According to one exemplary embodiment, the meal replacement includes a meal replacement base, and an appetite suppressant including a succulent plant extract. According to one exemplary embodiment, the succulent plant extract, which may be a dry powder, juice, pulp, or any other form is selected from the group consisting of Trichocaulon piliferum, Trichocaulon officinale, Hoodia gordonii, Hoodia currorii, Hoodia lugardii, and the Caralluma group of plants.

### DETAILED DESCRIPTION

The following description includes specific details in order to provide a thorough understanding of the novel meal replacement and appetite suppressant. Skilled artisan will understand, however, that the products and methods described below can be practiced without employing these specific details. Indeed, they can be modified and can be used in conjunction with products and techniques known to those of skill in the art.

A meal replacement product is provided herein having an appetite suppressing qualities, due to an added succulent plant extract that may be in the form of a powder, a juice, a pulp, or any other form, as a method of combating obesity in a human or “animal” by appetite suppression and thus lowering calorie intake. As used herein, “appetite suppressant” shall broadly be used to indicate activity, which tends to limit appetite and/or increase the sense of satiety, to reduce total calorific intake; this in turn tends to counteract obesity. Accordingly, this exemplary system and method extends to a method of treating, preventing or combating obesity in a human or non-human animal. According to one exemplary embodiment, the succulent powder/extract includes at least one succulent plant selected from the group including Trichocaulon piliferum, Trichocaulon officinale, Hoodia gordonii, Hoodia currorii, Hoodia lugardii, and the Caralluma group of plants. Other exemplary ingredients may be included with these powder/extracts to form the meal replacement. The meal replacement may be used instead of a meal, as a snack between the meals, or as a meal and/or a snack any time of the day. The term “animal” as used here extends to, but is not restricted to, companion animals, e.g. household pets and domesticated animals; non-limiting examples of such animals include cattle, sheep, ferrets, swine, camels, horses, poultry, fish, rabbits, goats, dogs and cats. Exemplary succulent powder/extracts will first be discussed, followed by a discussion of other optional ingredients.

The present exemplary system and method for providing a meal replacement formula includes a food item including an extract from a plant of the genus Trichocaulon, genus Hoodia, and/or from a plant of the genus Caralluma that comprises an appetite suppressant agent. Additionally, the meal replacement food item may also include a meal replacement base including a sugar substitute, protein, fiber, vitamins, minerals, and/or neutraceuticals, among other ingredients. Further details of each of the exemplary ingredients and their functions will be provided below.

According to one exemplary embodiment, a meal replacement formula contains an extract obtainable from a plant of the genus Trichocaulon, genus Hoodia, and/or from a plant of the genus Caralluma that comprises an appetite suppressant agent. Extracts from the genus Trichocaulon may be selected from the species Trichocaulon piliferum and Trichocaulon officinale. Extracts from plants of the genus Hoodia may be selected from the species Hoodia currorii, Hoodia gordonii and Hoodia lugardii. Extracts from the genus Caralluma may be selected from the species fimbriata, attenuata, tuberculata, adscendens, and indica. An extract of succulent plant may be present in any form and concentration, or as a fine powdered dry succulent plant or succulent juice.

Hoodia is a genus of succulent plants in the family Apocynaceae. The dry extract of Hoodia Gordonii may allow a person to reduce food consumption relatively effortlessly, with little or no side effects and with no substantial loss of vitality. Hoodia Gordonii has a similar but far greater effect than glucose on the brain's nerve cells yet without any of the calories. A molecule was isolated in the hoodia that sends messages to the brain signaling that caloric needs have been met thus satiating appetite, like does glucose. But contrary to glucose, this molecule does not generate any calories. Clinical studies have also shown that, due at least in part to this appetite suppressant, obese patients using the supplement can reduce their calorie intake by 30 to 40%, thus significantly improving their weight loss. Consequently, Hoodia Gordonii may aid in helping reestablish the true alimentary needs of a patient, when used in conjunction with a healthy, balanced, sufficient diet. Hoodia Currorii, which is eaten as a food, may also work as an appetite suppressant and to treat indigestion, hypertension, diabetes and stomach ache.

The Caralluma group of plants belongs to the Asclepiadaceae family and includes a number of species. Some of these species include but are not limited to: caralluma indica, caralluma fimbriata, caralluma attenuata, caralluma tuberculata, caralluma edulis, caralluma adscendens, caralluma stalagmifera, caralluma umbellata, caralluma penicillata, caralluma russeliana, caralluma retrospiciens, caralluma arabica and caralluma lasiantha. Some of the species are distributed throughout various parts of India. Caralluma plants are small, erect and fleshy. The Indian species of the Caralluma plant have four grooved stems that are almost round in shape. Plants of this species are generally devoid of leaves and form small flowers in a variety of dark colors. The Indian Caralluma plants frequently have pods that are erect, linear and about 2.5 cms in length and feel velvety to the touch. The thorns of Caralluma are soft. The species of Caralluma found in India are edible.

The medicinal properties of Caralluma includes carminative, febrifugal, anthelmintic, anti-rheumatic, anti-diabetic and anti-hyperglycaemic, anti-pyretic, anti-inflammatory, anti-nociceptive, and anti-oxidant effects. The Caralluma extracts have been also found to be appetite-suppressants as well as CNS stimulants.

The medicinal properties of Caralluma have been attributed to the glycosides contained therein. A glycoside is a condensation product obtained from a sugar and non-sugar compound and may have further components such as ring structures that are substituted or non-substituted. The glycosides contained in Caralluma belong to the pregnane group of glycosides.

Meal replacement if in a form of a proprietary shake mix, is preferably low in carbohydrate and low in sugar. The carbohydrate and sugar content of a mix can be reduced by replacing natural sugar sweeteners with sugar substitutes. Sugar substitutes do not significantly increase blood sugar levels and therefore do not count as net effective carbohydrates. The sugar substitutes used may include sugar alcohols, high intensity sweeteners, fiber sweeteners or combinations thereof.

Sugar alcohols are well-suited for use as sugar substitutes in low carbohydrate foods. Exemplary sugar alcohols can be up to about 95% as sweet as sucrose, and provide similar bulking properties as sucrose. Sugar alcohols are low-digestible carbohydrates because they are not fully absorbed from the intestines; thus, they provide fewer calories than sucrose and are less available for energy metabolism. Because of these properties, sugar alcohols do not cause the detrimental effects on diabetics that other carbohydrates cause. Examples of suitable sugar alcohols include, but are not limited to, mannitol, sorbitol, xylitol, lactitol, isomalt, maltitol, erythritol, D-tagatose, trehalose, isomalt and hydrogenated starch hydrolysates (HSH). Additionally, maltitol solution and other sugar alcohol solutions may be used to improve the texture, moisture content and crystallization properties of the low carbohydrate foods. In another aspect the sugar alcohol is isomalt, a sugar substitute produced by the isomerisation and catalytic hydrogenation of sucrose, with subsequent solidification and milling. Since it is based on sucrose, isomalt can be substituted for natural sugar or any other sugar alcohol in a 1:1 mass ratio.

Other sugar substitutes that can also be used include fiber sweeteners such as inulin and oligofructose. Inulin and oligofructose provide sweetness, as well as add bulk and creaminess. Inulin and oligofructose are thus useful as fat replacements, are good sources of soluble dietary fiber and are well-suited for diabetics because they do not increase the blood sugar level or insulin level due to their indigestible nature. Generally the fiber sweeteners can be used in addition to, or in place of, sugar alcohols. In one embodiment, the sugar substitute comprises inulin instead of sugar alcohols in order to further reduce or eliminate net effective carbohydrates. In another embodiment gums, such as guar gum, gum arabic, xanthan gum, carrageenan and pectin, can be added with the inulin to help improve the texture and to minimize or neutralize carbohydrates.

High intensity sweeteners can also be as sugar substitutes to reduce the net effective carbohydrate content of food while still providing a sweet taste. These high intensity sweeteners can be used in addition to, or in place of, sugar alcohols and fiber sweeteners. These sweeteners can be hundreds of times sweeter than sucrose, yet they contain many fewer calories than sucrose. High intensity sweeteners also have little to no effect on blood sugar levels. High intensity sweeteners include, but are not limited to, aspartame, alitame, neotame, cyclamates, saccharin, acesulfame, sucralose, neohesperidin dihydrochalcone, stevia sweeteners, glycyrrhizin, thaumatin, and the like, and mixtures thereof. Usually the amount of high intensity sweetener used is scant since it has such an intense sweetness.

The present exemplary meal replacement will support anyone wanting a healthy meal with limited carbohydrates, particularly persons seeking to control carbohydrate and sugar intake, lose weight, curb their appetite, and/or boost their energy level. It also offers a support to individuals with Type II Diabetes or those who are at risk to develop it. This is relevant not only as a method of treatment short-term weight control but also long-term weight maintenance. The meal replacement is also suitable for those on a low-carbohydrate diet.

The appetite suppressing meal replacement base may also include any kinds and amounts of protein, fibers, vitamins/minerals/fruits and vegetable extracts, neutraceuticals, botanicals and any other edible ingredients. For example, the meal replacement may contain protein material in order to provide additional nutritional benefits and an alternative from the traditional method for supplying protein in a diet. The protein material that can be used includes, but is not limited to, soy and soy isolates, whey and whey isolates, micro cross filtered whey isolates, wheat proteins (e.g., glutenir, gliadin, and gluten), isolated grain and vegetable proteins, egg white protein, protein isolates, and albumen isolates. Vegetable proteins include any vegetable in which proteins may be collected, whether condensed, accumulated or isolated. Examples of protein-providing vegetables include spelt, quinoa, amaranth, buckwheat, black rice and the like. The protein material can be any single type of protein, or may be a blend of various proteins that provides unique texture, flavor, durability and strength characteristics to the ingesting medium based on the composition of the blend.

Fiber may also be used to give good mouth feel and to reduce the net effective carbohydrate content. The fiber that may be used in the present system and method includes plant-based fibers, such as those derived from vegetables, legumes, fruits and grains. The fiber may be soluble or insoluble, and includes, but is not limited to, non-starch polysaccharides, resistant starches, modified starches, lignin and fructo-oligosaccharides. Although fructo-oligosaccharides are not technically fibers, they are classified as dietary fiber since they are not absorbed in the intestine, and therefore have a zero net carbohydrate effect. Examples of suitable fructo-oligosaccharide fiber include inulin and oligofructose. A fiber rich diet may also be desirable to help alleviate or prevent conditions including constipation, diverticulosis, colon and rectal cancer, heart disease, breast cancer, diabetes and obesity.

The meal replacement can also act as an ideal carrier for vitamins and minerals. Examples of vitamins that can be added to the ingested medium include, but are not limited to, vitamin A, vitamin B₁₂, vitamin C, vitamin D, vitamin E, vitamin K, para-aminobenzoic acid, Vitamin B₂ (riboflavin), Vitamin B₆, niacin, inositol, biotin, folic acid, choline, and vitamin B₁. Examples of minerals that can be added include, but are not limited to, magnesium, iron, zinc, copper, manganese, sodium, potassium, calcium, selenium, chromium, molybdenum, chlorine, fluorine, phosphorus, sulfur, and iodine. The meal replacement can also contain botanicals (neutraceuticals) including, but not limited to, bilberry, cascara, cat's claw, cayenne, cranberry, devil's claw, dong quai, echinacea, evening primrose oil, feverfew, garlic, ginger, ginkgo, Asian ginseng, Siberian ginseng, goldenseal, gotu kola, grape seed, green tea, hawthorn, kava, licorice, milk thistle, noni, saw palmetto, St. John's wort, valerian, melatonin, damiana, yerbe mate, guarana, Rhodiola Rosea, camu-camu berries, Wolfberries, Acai, borojoa patinoi, and the like.

The meal replacement can be in the form of a dry powder mix, to be reconstituted with water, milk, or juice, or in ready to drink packaging, any edible/drinkable form, any form of nutritional beverages, puddings, confections (i.e., candy), ice cream, frozen confections and novelties, hot chocolate, coffee, tea, juice, confectionaries, chocolates, probiotic drinks/yogurt/kefir, nutritional/energy bars, soups, breads/cereals, chips and etc.

In another embodiment, the ingredients of the nutritional supplement can be administered separately, such as by incorporating certain components (e.g., bitter tasting ones) into a capsule or tablet and the remaining ingredients are provided as a powder or nutritional bar.

The appetite suppressing extract used in meal replacement could be prepared from plant material such as the stems and roots of said plants of the genus Trichocaulon or of the genus Hoodia. The genus Trichocaulon and the genus Hoodia include succulent plants growing in arid regions such as are found in Southern Africa. Besides possessing appetite suppressing and thirst quenching qualities, hoodia also possesses a sense of well being, and, anecdotal accounts regarding its aphrodisiac properties.

The present exemplary system and method extends also to a composition or formulation having appetite suppressant activity, in which the active ingredient is a synthetically produced version of Hoodia Gordonii, a member of the Caralluma group or a derivative or analogue thereof.

The present exemplary system and method extends also to a foodstuff or a beverage containing an effective quantity of the appetite suppressing Hoodia species, or its derivatives or analogues as set out before, to have an appetite suppressant effect when ingested. If in a powder form, which is reconstituted with water, milk or juice, the powder is to be mixed in any kind of ways, as in an electric blender or by simply shaking/stirring the powder and the liquid in a container. Alternatively, the powder could be agglomerated to facilitate reconstitution without an electric food blender.

A weight control meal replacement is formulated such that one serving of the beverage/bar/chocolate/soup/etc, acts as a meal replacement and provides any amount of United States Recommended Daily Allowance (US RDA) for protein, minerals and vitamins, fiber, and any amount of calories.

The preceding description has been presented only to illustrate and describe the present method and products. It is not intended to be exhaustive or to limit the disclosure to any precise form disclosed. Many modifications and variations are possible in light of the above teaching. It is intended that the scope of the present system and method be defined by the following claims.

What is claimed is:

1. A meal replacement, comprising:
a meal replacement base, and
an appetite suppressant including a succulent plant extract.

2. The meal replacement of claim 1, wherein said succulent plant extract is selected from the group consisting of Trichocaulon piliferum, Trichocaulon officinale, Hoodia gordonii, Hoodia currorii, and Hoodia lugardii.

3. The meal replacement of claim 1, wherein said succulent plant extract is selected from the Caralluma group of plants.

4. The meal replacement or claim 3, wherein said succulent plant extract is selected from the group consisting of caralluma indica, caralluma fimbriata, caralluma attenuata, caralluma tuberculata, caralluma edulis, caralluma adscendens, caralluma stalagmifera, caralluma umbellata, caralluma penicillata, caralluma russeliana, caralluma retrospiciens, caralluma Arabica, and caralluma lasiantha.

5. The meal replacement of claim 1, wherein said meal replacement base includes a sugar substitute.

6. The meal replacement of claim 5, wherein said sugar substitute comprises a sugar alcohol.

7. The meal replacement of claim 6, wherein said sugar alcohol includes at least one of mannitol, sorbitol, xylitol, lactitol, isomalt, maltitol, erythritol, D-tagatose, trehalose, isomalt hydrogenated starch hydrolysates, or isomalt.

8. The meal replacement of claim 5, wherein said sugar substitute includes a fiber sweetener.

9. The meal replacement of claim 8, wherein said fiber sweeter includes at least one of inulin and oligofructose.

10. The meal replacement of claim 9, and further comprising at least one of guar gum, gum arabic, xanthan gum, carrageenan, and pectin.

11. The meal replacement of claim 9, wherein said sugar substitute includes a high intensity sweetener.

12. The meal replacement of claim 1, wherein said meal replacement base includes at least one protein selected from a group consisting of soy and soy isolates, whey and whey isolates, micro cross filtered whey isolates, wheat proteins, glutenir, gliadin, gluten, isolated grain and vegetable proteins, egg white protein, protein isolates, and albumen isolates.

13. The meal replacement of claim 1, wherein said meal replacement base includes at least one vegetable protein.

14. The meal replacement of claim 1, wherein said meal replacement base includes at least one protein-providing vegetables selected from a group consisting of spelt, quinoa, amaranth, buckwheat, and black rice.

15. The meal replacement of claim 1, wherein said meal replacement base includes at least one of vitamin A, vitamin B₁₂, vitamin C, vitamin D, vitamin E, vitamin K, para-aminobenzoic acid, Vitamin B₂ (riboflavin), Vitamin B₆, niacin, inositol, biotin, folic acid, choline, and vitamin B₁, magnesium, iron, zinc, copper, manganese, sodium, potassium, calcium, selenium, chromium, molybdenum, chlorine, fluorine, phosphorus, sulfur, and iodine.

16. The meal replacement of claim 1, wherein said meal replacement base includes at least one of bilberry, cascara, cat's claw, cayenne, cranberry, devil's claw, dong quai, echinacea, evening primrose oil, feverfew, garlic, ginger, ginkgo, Asian ginseng, Siberian ginseng, goldenseal, gotu kola, grape seed, green tea, hawthorn, kava, licorice, milk thistle, noni, saw palmetto, St. John's wort, valerian, melatonin, damiana, yerbe mate, Rhodiola Rosea, camu-camu berries, Wolfberries, Acai, borojoa patinoi and guarana.

17. The meal replacement of claim 1, wherein said meal replacement is in the form of a dry powder mix, to be reconstituted with liquid.

18. The meal replacement of claim 1, wherein said meal replacement is in ready to drink packaging.

19. The meal replacement of claim 1, wherein said meal replacement is in the form of at least one of a group consisting of beverages, puddings, confections, ice cream, frozen confections and novelties, hot chocolate, coffee, tea, confectionaries, chocolates, nutritional/energy bars, soups, breads/cereals, probiotic drinks/yogurt/kefir, and chips.

20. The meal replacement of claim 1, wherein said meal replacement base includes a first portion of the appetite suppressant is in a form of a tablet/capsule/gel cap and a second portion is configured to be administered separately from said first portion.

21. A meal replacement, comprising:
a meal replacement base having a sugar substitute, a protein, fiber, vitamins and minerals; and
an appetite suppressant having a succulent plant extract is selected from the group consisting of Trichocaulon piliferum, Trichocaulon officinale, Hoodia gordonii, Hoodia currorii, Hoodia lugardii and the Caralluma group of plants.

22. The meal replacement of claim 21, wherein said meal replacement is in a ready to drink form.

23. The meal replacement of claim 21, wherein said meal replacement is in a powdered form.

24. The meal replacement of claim 21, wherein said meal replacement is in a bar form.

25. A method of forming a meal replacement, comprising:
providing a meal replacement base;
adding an appetite suppressant to said meal replacement base, said appetite suppressant including a succulent plant extract selected from the group consisting of Trichocaulon piliferum, Trichocaulon officinale, Hoodia gordonii, Hoodia currorii, Hoodia lugardii and the Caralluma group of plants.
